(19) United States
(12) Patent Application Publication
(10) Pub. No.: US 2014/0039278 A1
(43) Pub. Date: Feb. 6, 2014

(54) BARIATRIC INSTRUCMENT OR ACCESSORY WITH SENSORS

(71) Applicant: Allergan, Inc., Irvine, CA (US)

(72) Inventor: Janel A. Birk, Oxnard, CA (US)

(73) Assignee: Allergan, Inc., Irvine, CA (US)

(21) Appl. No.: 14/049,351

(22) Filed: Oct. 9, 2013

Related U.S. Application Data
(63) Continuation of application No. 13/510,479, filed on May 17, 2012, now abandoned.
(60) Provisional application No. 61/263,330, filed on Nov. 20, 2009.

(51) Int. Cl. A61B 5/00; A61B 5/11; A61F 5/00; A61B 5/01; A61B 5/1473; A61B 5/03; A61B 5/145

(52) U.S. Cl. CPC A61B 5/4848; A61B 5/037; A61B 5/42; A61B 5/1107; A61B 5/14539; A61B 5/01; A61B 5/1473; A61B 5/14532; A61B 5/14546; A61B 5/0002; A61F 5/0079; A61F 5/003; A61F 5/005; A61B 5/4836
USPC 600/301; 600/300

(57) ABSTRACT

A bariatric instrument or accessory with sensors 28 used for sensing or monitoring parameters related to a patient during the placement, adjustment or in vivo duration of a bariatric or intraluminal device 10 such as a balloon, stent, feeding tube or other. The instrument or accessory may sense parameters from direct patient contact, non-patient contact such as intraluminal data, or indirect patient contact. The instrument 20 could be used during placement and adjustment of a device. The accessory 30 could be attached to a bariatric or intraluminal device 10 for the placement or adjustment of a bariatric device, or could be left on the bariatric device for in vivo placement.

### RELATED APPLICATIONS

This application is a continuation of U.S. patent application Ser. No. 13/510,479, filed May 17, 2012, which is a national stage application under 35 USC §371 of PCT Patent Application No. PCT/US2010/057530, filed Nov. 19, 2010, which claims the benefit of U.S. Provisional Patent Application No. 61/263,330 filed Nov. 20, 2009, the entire disclosure of each of these applications being incorporated herein by this reference.

### TECHNICAL FIELD

This invention relates to a bariatric or intraluminal device for weight loss, and ancillary items such as sizing, and monitoring.

### BACKGROUND

Obesity has been steadily increasing worldwide and poses serious health risks, which if untreated, can become life threatening. There are various methods for reducing weight such as diet, exercise, and medications but often the weight loss is not sustained. Significant advances have been made in the surgical treatment of obesity. Surgical procedures such as the gastric bypass and gastric banding have produced substantial and lasting weight loss for obese patients. These procedures and products have been shown to significantly reduce health risks over time, and are currently the gold standard for bariatric treatment.

Although surgical intervention has been shown to be successful at managing weight loss, both procedures are invasive and carry the risks of surgery. Gastric bypass is a highly invasive procedure which creates a small pouch by segmenting and/or removing a large portion of the stomach and rerouting the intestines permanently. Gastric bypass and its variations have known complications. Gastric banding is an invasive procedure which creates a small pouch in the upper stomach by wrapping a band around the stomach to segment it from the lower stomach. Although the procedure is reversible, it also carries known complications.

Less invasive or non-invasive devices that are removable and capable of significant weight loss are desirable. Using scientific means to properly place or size these non-invasive bariatric devices at the time of placement provides a greater potential that the device or surgical procedure will perform as intended with a reduced level of complications. This provides a higher level of confidence for the physician and improved device acceptance by the patient.

### SUMMARY

The bariatric instrument or accessory described herein is an intraluminal instrument or accessory with a sensor to be used with a bariatric or intraluminal device or procedure to monitor one or more parameters inside the gastrointestinal (GI) track including the esophagus, stomach and intestines. The parameter could be pressure, force, peristalsis, pH, motion, tension, pH, temperature, chemical, glucose, hormonal, or other. The instrument is preferably separate from the bariatric or intraluminal device and will typically be constructed with a shaft for placement down the esophagus and possibly an arm for manipulation. In contrast, the device accessory is preferably attached to or contacting the bariatric or intraluminal device and may be removed after the device is placed.

The instrument or device accessory could be used as a guide during placement of a bariatric device to monitor placement, performance, adjustments or other data as needed. The bariatric instrument or accessory is used when placing a bariatric or intraluminal device or performing a bariatric surgical procedure that induces weight loss by a variety of weight loss mechanisms. These weight loss mechanisms could include gastric intake restriction, with devices such as an adjustable gastric band, gastric bypass, sleeve gastrectomy, or gastric valves. The instrument could be used to ensure that the band or bypass anastomosis is placed in the proper location and that the band is adjusted to a proper volume. The weight loss mechanism could also include space occupying devices such as an inflatable intragastric balloon or other similar devices, where the instrument is used to ensure proper fill volumes are achieved to engage the stretch receptors and to lose weight. The weight loss mechanism could also include reduced gastric emptying, gastric sleeves or intestinal sleeves for malabsorption. The weight loss mechanism could also include devices that distend the stomach, apply pressure to the stomach or engage a satiety response, such as applying pressure to the cardia of the stomach to engage a neurohormonal response. In these cases, the instrument could be used to gather data on where the device is placed to ensure the force is adequate to engage the satiety response, but not so great that it induces nausea. The instrument or accessory equipped with sensors can gather placement or adjustment data to customize the placement and/or fit to the patient for improved long term performance.

To ensure that the bariatric or intraluminal device or procedure has applied an appropriate amount of force or pressure within the gastrointestinal track, the instrument could contain one or more sensors to measure one or more patient parameters such as force or pressure. Such a sensor could be in direct contact with the patient, could have intermittent contact with the patient, could not be in direct contact the patient to gather intraluminal pressure or other parameters or could be any combination of the above. The sensor could sense a variety of parameters such as force, pressure, stress, peristalsis, motion, pH, temperature, chemical, glucose, or other appropriate parameters, or various parameter combinations. Preferably, such parameter(s) could be read real time during the implant surgery, placement, medical intervention, operation, or adjustment.

The sensor could be wireless or could be wired. Whether a wired or wireless sensor is used, the external controller may have the capability to gather and record data. The external controller may also contain the ability to perform analysis of the collected data for further diagnostic capabilities. The external controller may have the capability to gather the data and display it in a variety of presentations. It may display raw data, averages, or it could analyze the data and diagnose a generalized state as being appropriate or inappropriate. For example, an inappropriate state might be displayed with a red light while an appropriate state might be indicated with a green light. Similarly, the display could be shown in a lighted bar graph where a more appropriate state is indicated by more bars and a less appropriate state is indicated by less bars. Where a wired sensor is used, the external controller could be connected and integrated into the instrument for reading the parametric data. Where a wireless sensor is used, an external controller could be connected and integrated into the instrument or the external controller could be a separate unit for ease of handling or to increase the display size without encumbering the instrument use. Further description of the wireless and wired sensors is included below.

The instrument or device accessory could contain one sensor or multiple sensors. It could also employ an array of sensors that are positioned on top of or integrated into a thin, flexible sheet or element. This element could take a variety of shapes including a strip, disk, frusto-cone, sphere, a portion of any of these or other. Where an array of sensors is used, the display may show a 2D or 3D color plot or graphical representation of the pressure mapping across the sensor array. A variety of visual displays could be used to represent the state of the device condition. The sensor arrays could be located on a single arm or multiple arms. The single arm could take the form of a loop, a curved wire, a spiral, cylinder, cone or multiples of these, or other shapes and multiples, to cover a region of interest. The instrument arm or arms could articulate to allow for manipulation for ideal positioning of the instrument during the procedure. The instrument could have a narrow cross-section to allow it to fit down the working channel of a gastroscope. Alternatively, it may require a larger sizing for additional features such as articulating arms, but would preferably be sized small enough to fit down the esophagus next to the gastroscope, and long enough for proper manipulation outside the body. Where there are expanding or articulating features, the instrument or accessory may have adequate ability to collapse into a long narrow profile to facilitate placement down the esophagus. The instrument would preferably be smooth and contoured to reduce the potential for tissue irritation.

The sensor could be in indirect contact with the patient such as being contained inside of a sizing balloon or inside of a tube where the GI tract contacts the balloon or tube and the sensor is inside. The sensor could be sealed inside the balloon or tube to measure the pressure or force variant or it could monitor other conditions in an unsealed condition.

The instrument could be reusable or disposable. After the device placement, adjustment, or procedure was completed, the instrument would be removed.

The instrument or device accessory could be made of many different materials or combinations of materials. For an instrument, the materials would be acid resistant for transient contact with the stomach for single or repeat use. For a device accessory that is intended to remain on the device, the accessory may need more acid resistant properties.

Elements of the device could be made of Nitinol, shape memory plastics, shape memory gels, stainless steel, superalloys, titanium, silicone, elastomers, teflons, polyurethanes, polynorborenes, styrene butadiene co-polymers, cross-linked polyethylenes, cross-linked polycyclooctenes, polyethers, polyacrylates, polyamides, polysiloxanes, polyether amides, polyether esters, and urethane-butadiene co-polymers, other polymers, or combinations of the above, or other suitable materials.

### BRIEF DESCRIPTION OF DRAWINGS

FIG. 1A depicts a side view of an embodiment of the bariatric instrument of the present invention and a cross-section of a bariatric device located within a cross-section of a stomach.

FIG. 1B depicts a side view of an embodiment of the bariatric instrument of the present invention and a cross-section of a bariatric device located within a cross-section of a stomach.

FIG. 1C depicts a display of a pressure map from a sensor array.

FIG. 1D depicts an embodiment of the present invention with an integrated controller and display, as well as a separate controller unit option, demonstrated during use with a patient.

FIG. 1E depicts an embodiment of the present invention with a wireless external controller used near the patient.

FIG. 2 depicts a side view of an embodiment of the bariatric instrument of the present invention located within a cross-section of a stomach.

FIG. 3 depicts a side view of an embodiment of the bariatric instrument of the present invention and a cross-section of a bariatric device located within a cross-section of a stomach.

FIG. 4A depicts a side view of an embodiment of the bariatric device accessory of the present invention and a cross-section of a bariatric device located within a cross-section of a stomach.

FIG. 4B depicts a top view of the embodiment in FIG. 4A.

FIG. 4C depicts a top view of an alternative to the embodiment in FIG. 4A.

FIG. 5 depicts a side view of an embodiment of the bariatric accessory of the present invention located within a cross-section of a stomach.

FIG. 6 depicts a side view of an embodiment of the bariatric accessory of the present invention located within a cross-section of a stomach.

### DETAILED DESCRIPTION OF THE INVENTION

The detailed description set forth below in connection with the appended drawings is intended as a description of presently-preferred embodiments of the invention and is not intended to represent the only forms in which the present invention may be constructed or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

### Embodiment with Direct Tissue Contact

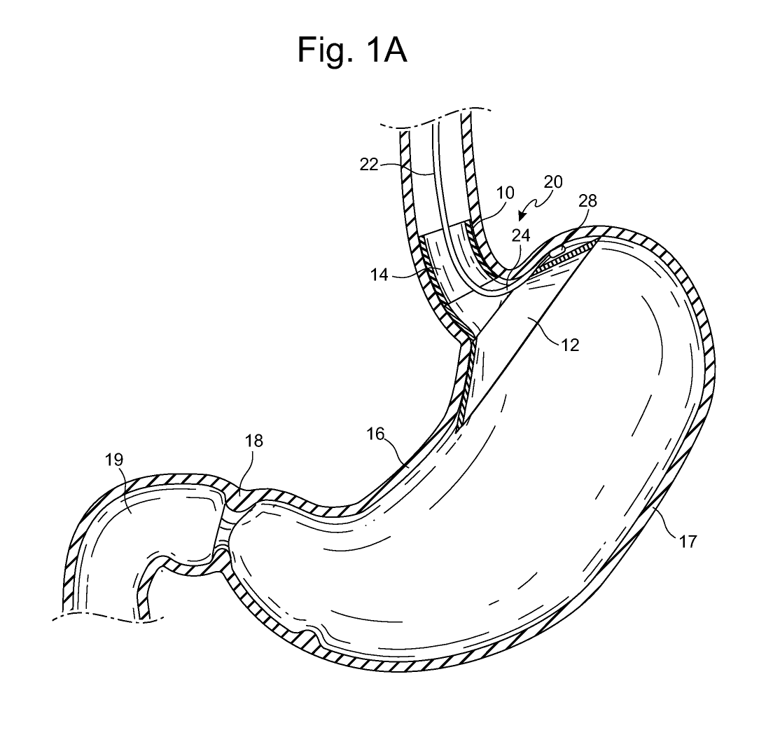

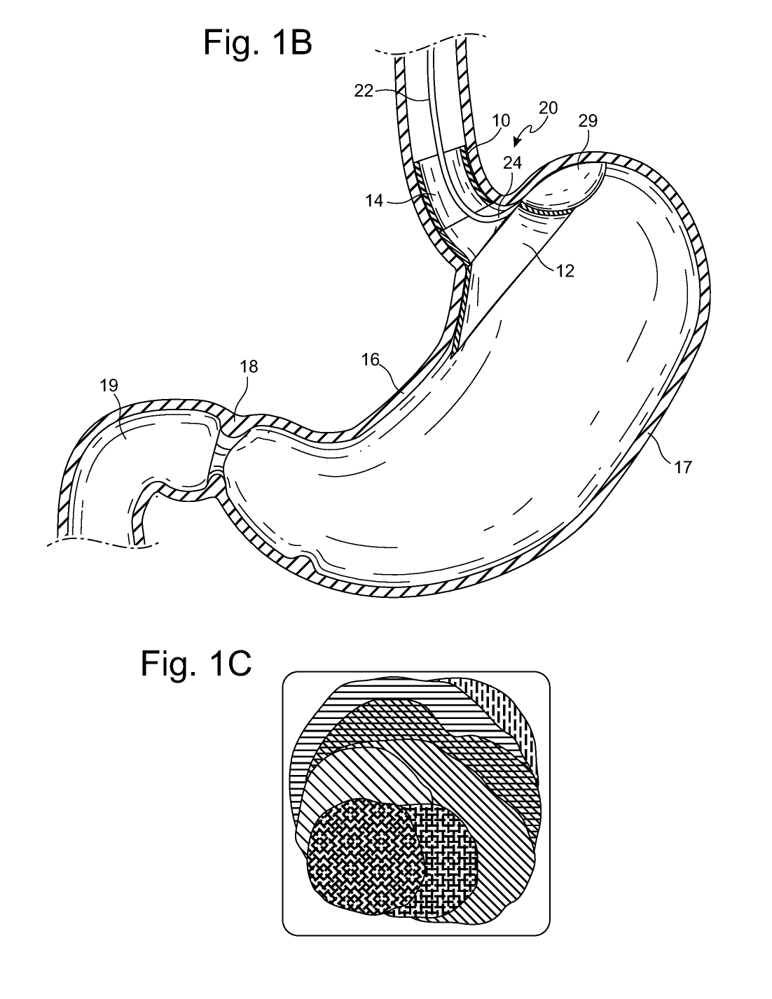

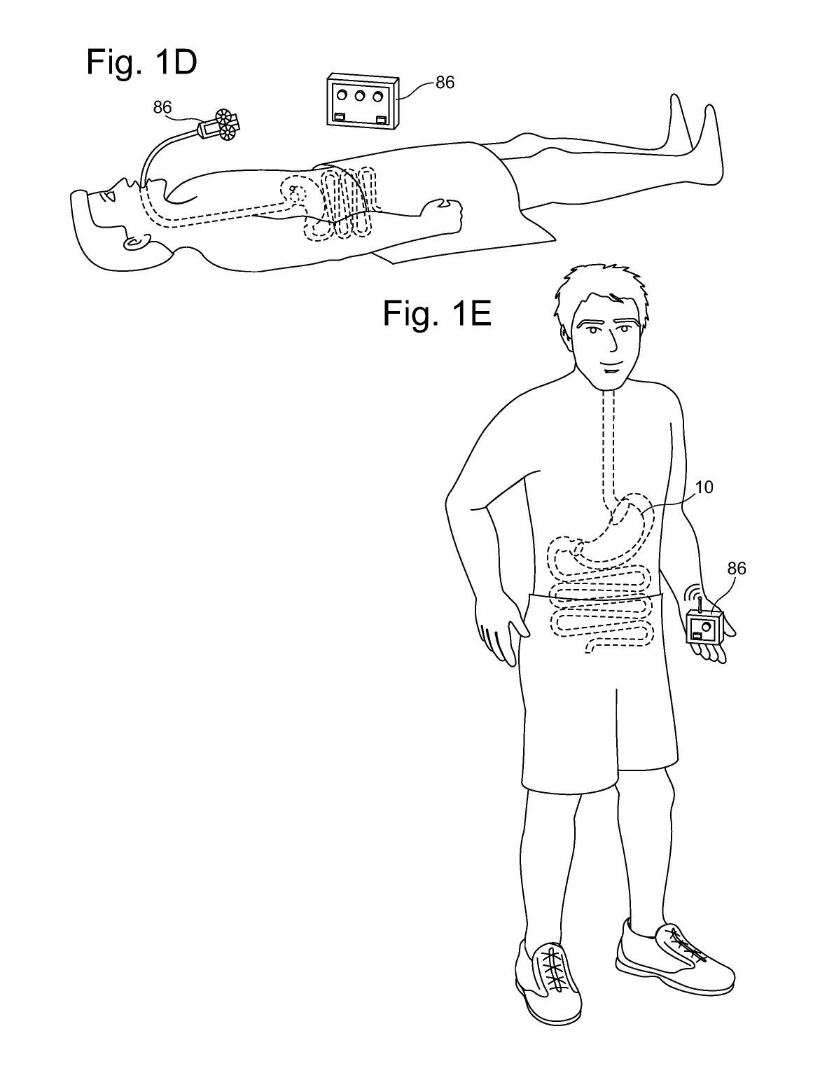

In the most basic embodiment, the instrument 20 could contain a central shaft 22 that seamlessly connects to a single arm 24 which contains one or more sensors 28. This embodiment has sensors that are in direct tissue contact with the patient. For example, an intragastric bariatric device 10 is placed that has a cardiac element 12 which delivers direct force to the proximal cardiac portion of the stomach, and an esophageal element 14 that delivers force to the esophagus. See FIG. 1A. The instrument 20 is positioned between the bariatric device 10 and the patient tissue to gather data on how much pressure or force the bariatric device 10 is applying to the patient. In this embodiment, a single arm 24 is used to curve and angle the sensor 28 into a location of interest. This instrument 20 could have an articulating arm which could bend or curve in one or more planes similar to a gastroscope or it could be pre-curved and straightened by a central guide wire which could be removed once the instrument 20 was inside the stomach to allow it move into position. Similarly, the instrument 20 could be made of shape memory or super elastic material that can be straightened for insertion and then curve into shape once inside the stomach. The instrument 20 could also be straight and flexible, and could pass through the central channel of a gastroscope to allow the gastroscope to provide the means of articulation to move the instrument 20 into a preferred location. The instrument 20 could also be attached to the outside of a gastroscope to allow it to be manipulated into position.

As an alternative, the single arm 24 could have an array of sensors integrated into a sheet or shaped surface to better characterize the pressure profile of the contact surface. See FIGS. 1B and 1C. FIG. 1B shows an instrument 20 with a disk shaped sensor array to create a pressure mapping along the proximal cardia. Although the sensor array 29 shown is disk shaped, the array could take a variety of shapes including a strip, a ring, frusto-cone, sphere, or a portion of any of these or other. FIG. 1C shows a possible example of a 2D pressure mapping from the sensor array in grayscale for purposes of this application. Preferably, this display would be in color. This pressure profile could be mapped in a 2D or 3D color plot for improved visualization. An example of a sensor array is available from Tekscan where they can control the density of sensors and have high measurement accuracy. These sensors are available in very thin flexible sheets and a variation of those sensor arrays could be applied towards this type of instrument. An alternative would be a sensor available from Vista Medical where they also offer sensor arrays in a thin flexible sheet. The sensor array sheet could be collapsed or coiled into the instrument shaft 22 for placement down the esophagus and then could be deployed open once in the esophagus, stomach or intestines. The array could then be retracted back into the instrument shaft 22 for retrieval up the esophagus. Software to map, analyze and display the sensor array measurements may be required for use with a separate computer or in an external controller 86 with a microprocessor.

The instrument 20 may have a display to monitor the real-time pressure or force being exerted to guide the physician during placement, and this display could be external to the patient. The display could be small and integrated into the instrument 20 or it could be more sophisticated and contained in a separate unit. FIG. 1D shows an example where the external controller with display is integrated into the instrument 20. FIG. 1D also shows an alternative embodiment where the instrument could plug into a separate unit for a larger or more sophisticated controller and display. FIG. 1E shows an external controller with display that could be used with either an instrument 20 or a device accessory 30 where wireless communication is being utilized. While monitoring the data, the physician could alter the orientation, location and subsequent force on the bariatric device 10, to ensure it is within an ideal range prior to fixing the bariatric device 10. Alternatively, the instrument 20 could have the ability to collect and analyze the data with an algorithm to determine whether the bariatric device 10 was in the ideal orientation. At present, the bariatric device 10 is placed using the physician's best judgment, without actual diagnostic data on the force or pressure being exerted. With an instrument that can gather parametric data, the placement will be guided on data instead of judgment.

Preferably, the sensors would be adapted to accurately monitor very low pressures with fine resolution, low hysteresis and would be adapted for tissue contact. The sensors could have a very small surface contact area or could have a wider surface contact area.

The instrument 20 could contain wireless or wired sensors 28. Where wired sensors 28 are used on the instrument 20, the wires used to transmit data could be contained inside an instrument shaft 22, and data could be sent directly from the sensor to the display for monitoring, or to a microprocessor for analysis and then to the display. The microprocessor or external controller could be integrated directly into the instrument or the instrument could plug into a separate external controller 86 as both shown in FIG. 1D. Where wireless sensors 28 are used on the instrument 20, an external controller 86 would be used that could remotely send and receive signals via telemetry from the sensor as shown in FIG. 1E. The external controller 86 could display the data for monitoring or could contain a microprocessor for analysis and then display the data. In one embodiment, a wireless or wired sensor 28 may be used on the instrument 20 to communicate with a separate external controller 86 unit. It may be desirable to control the sensor from the external controller unit. The external controller 86 unit could send a command to the sensor to query it to start gathering data. The external controller could also send a separate or simultaneous command to send data. The sensor 28 would receive the command from the external controller and then transmit or respond to collect and/or send data. When sufficient data was received, a command could be sent from the external controller 86 to the sensor 28 to tell the sensor to stop gathering and/or sending data. These same communication features could be applied towards the accessory 30 embodiments as well.

In addition, the sensor 28 and or memory module of the instrument 20 or accessory 30 may be communicatively coupled with a transmitter, a receiver, or both, to allow communication of data or other information with outside receivers and transmitters. The transmitter may transmit signals received from the sensor, or signal data stored in the memory module.

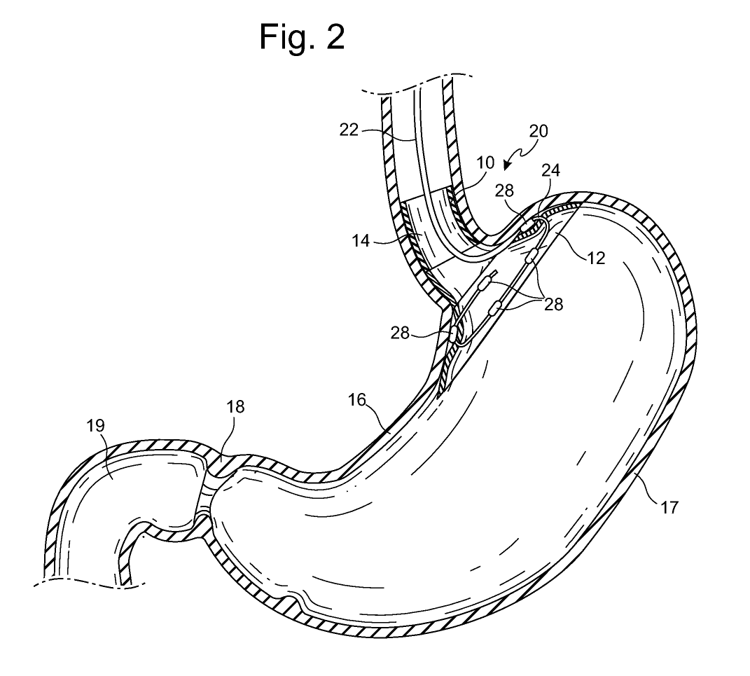

In another embodiment, the instrument 20 could have a single arm 24 with multiple sensors 28 or an array of sensors 29. FIG. 2 depicts one such an embodiment used with a bariatric device 10. In this embodiment, a central shaft 22 is connected to a single arm 24 and is used to curve into a shape to better match the bariatric device 10 being placed and to monitor a location of interest. FIG. 2 shows where a conical cardiac element 12 of the bariatric device 10 is being placed at the cardia or upper stomach. The instrument 20 is curved into a conical spiral that matches the shape of the cardiac element 12, which allows the sensor to monitor the pressure or force being applied by the bariatric device 10 to the cardia. This embodiment of the instrument 20 could have an articulating arm or it could be pre-curved into a general shape to best suit the interface between the bariatric device 10 and the anatomy. The instrument 20 could have a rigid, semi-rigid, or flexible construction or a combination of any of the above. The single arm 24 could be constructed with multiple links that articulate or it could be made from soft flexible polymer tubing that is positioned by a pre-shaped central guide wire. Conversely, the polymer tubing could be pre-shaped, and could be straightened by a central guidewire. Similarly, the instrument with a single arm 24 could be made of shape memory or super elastic material that can be straightened or constrained for insertion and then curve into a preferred shape once inside the stomach. The sensors 28 could also be placed onto soft flexible strips of material such as silicone, PTFE, FEP, or other polymers which are all connected to an instrument shaft 22 for control and retrieval. The sensors 28 could be supported and guided by the bariatric device 10. After the bariatric device 10 is placed, the central shaft 22 could be used to retrieve the sensors 28. There could be features on the bariatric device 10 that the instrument 20 could attach to or other features such as a temporary magnet, mechanical feature, biodegradable adhesive or other feature that could be used to temporarily connect the instrument to the bariatric device 10.

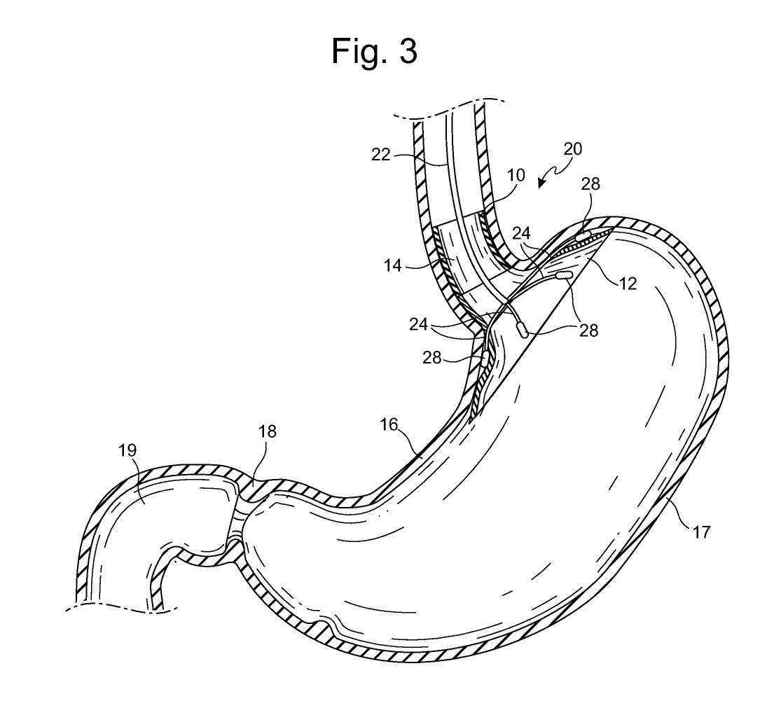

In another embodiment, the instrument sensor 28 may have direct tissue contact with the patient. For example, an intragastric bariatric device 10 is placed that has a cardiac element 12 which delivers direct force to the proximal cardiac portion of the stomach, and an esophageal element 14 that delivers force to the esophagus. See FIG. 3. In this embodiment, the instrument 20 has a central instrument shaft 22, multiple arms 24 and multiple sensors 28 to monitor the distribution of force around cardiac element 12 while it is being placed. As the physician is holding the bariatric device 10 in place prior to fixing it to the tissue, the instrument sensors 28 will be between the bariatric device 10 and the patient's tissue to monitor the force or pressure that the bariatric device 10 is placing on the patient. Each arm 24 may contain a single sensor, multiple sensors 28 or an array of sensors 29. Each arm 24 may also be able to be manipulated separately or they could articulate in conjunction to create a cone or spherical shape or other to better match the bariatric device geometry. FIG. 3 shows four arms 24 and four sensors 28 that are in direct patient contact. In an alternative embodiment, some of the sensors could be in direct contact while others are not in contact, but could gather intraluminal pressure data.

Although several of the embodiments above describe use with bariatric devices, the instrument 20 may also be applicable to non-bariatric devices such as stents, feeding tubes, or other intraluminal non-bariatric devices where parameter detection is desirable. For the purposes of the claims, the term “intraluminal device” shall include both bariatric and non-bariatric intraluminal devices.

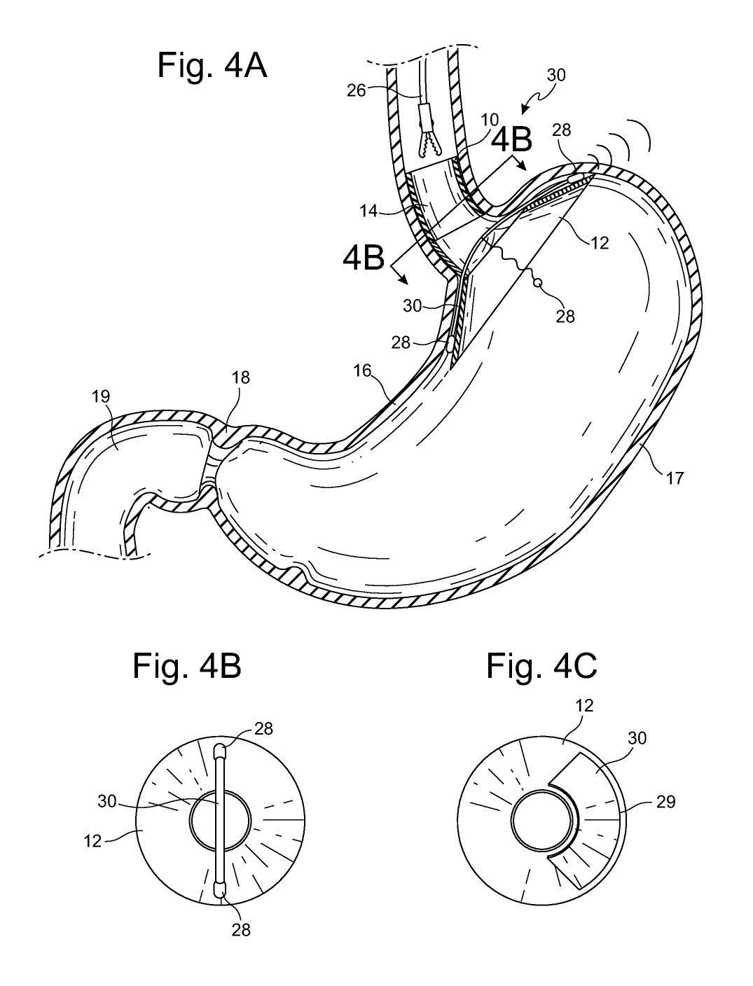

Use of wireless sensor 28 would allow for an alternative to an instrument 20, in the form of an accessory 30 that is temporarily or permanently attached to the bariatric device 10 for its placement. See FIGS. 4A and 4B. The accessory 30 is a modular feature and it could be attached to the bariatric device 10 prior to placement in the stomach or after placement into the stomach. The accessory 30 could be applied to several different types of devices including non bariatric devices such as stents, feeding tubes or other devices. After the placement of the bariatric device, the accessory 30 could be retrieved by standard instrumentation 26 for retrieval up the esophagus. This drawing shows a single arm of an accessory 30 with multiple sensors 28. FIG. 4A shows that two of the sensors 28 are in direct patient contact and a third sensor is tethered to gather intraluminal pressure. FIG. 4B shows a top view of the accessory 30. The accessory 30 could be temporarily attached to the bariatric device 10 or just draped on top of the bariatric device 10 for retrieval later. In this embodiment, wireless sensors 28 are used, and the accessory 30 is retrieved by standard instruments 26 after the placement is complete.

There could be attachment element features on the bariatric device 10 or other intraluminal device that the accessory 30 could attach to or other features such as a magnet, mechanical feature, biodegradable adhesive or substrate or other features could be used as attachment elements to temporarily connect the accessory 30 to the bariatric or intraluminal device 10. The accessory 30 could also be designed with the intent to disassociate from the bariatric device 10 and pass naturally through the intestines to eliminate the need for removal. For example, the accessory 30 could use an attachment element of biodegradable materials or materials that break down in stomach acid to allow the 30 to disassociate and pass. In such case, the accessory 30 may be attached to the bariatric device 10 for a short period of time, but not intended for long term placement. In other embodiments, the attachment element could be adapted for permanent or semi-permanent attachment to the bariatric or intraluminal device, which might include long-lasting adhesives, mechanical connections, Velcro, or other suitable attachment mechanisms.

In the accessory 30, the wireless sensor 28 could collect and store data over time which could then be wirelessly transmitted to an external controller under physician or patient control. The storage could take place in a memory module, which may be integrated with the sensor or separate. The memory module may use solid state or flash memory, or other suitable data memory devices.

The sensor 28 could be passive or active. In the case where the sensor 28 is active, it would require a power source such as an implantable battery. This would allow the sensor 28 to query on a routine basis and store the data in a memory module. The data could also be retrieved and down loaded, preferably by an external controller, but also by any other suitable device capable of retrieving the signals. Alternatively, the sensors 28 could be passive and require power to query the sensors 28 from an external source such as induction. The external controller could have the ability to inductively power the sensor remotely from outside the body, and remotely communicate with the sensor(s) 28 and/or the memory module to collect and download data. Alternatively or in addition to powering the sensors 28, induction may be used to power an on-board signal transmitter of the accessory 30 so that the stored and/or present values generated by the sensors 28 may be retrieved by the external controller or other suitable device.

The accessory 30 could also contain a sensor array 29 to better characterize the interface between the stomach and the bariatric device 10. For example, the sensor array 29 could substantially match the surface or a portion of the surface of the bariatric device 10. FIG. 4C shows a sensor array 29 in the shape of a portion of a frusto-cone and is attached to the outside of the frusto-cone bariatric device 10. Alternatively, the array could be a full frusto-cone or a larger portion of frusto-cone. For an intragastric balloon 16 the array could be a portion of a sphere, a disk or other shape. Such a sensor array could contain an antenna and have wireless transmission with all the same features for a wireless system as previously described.

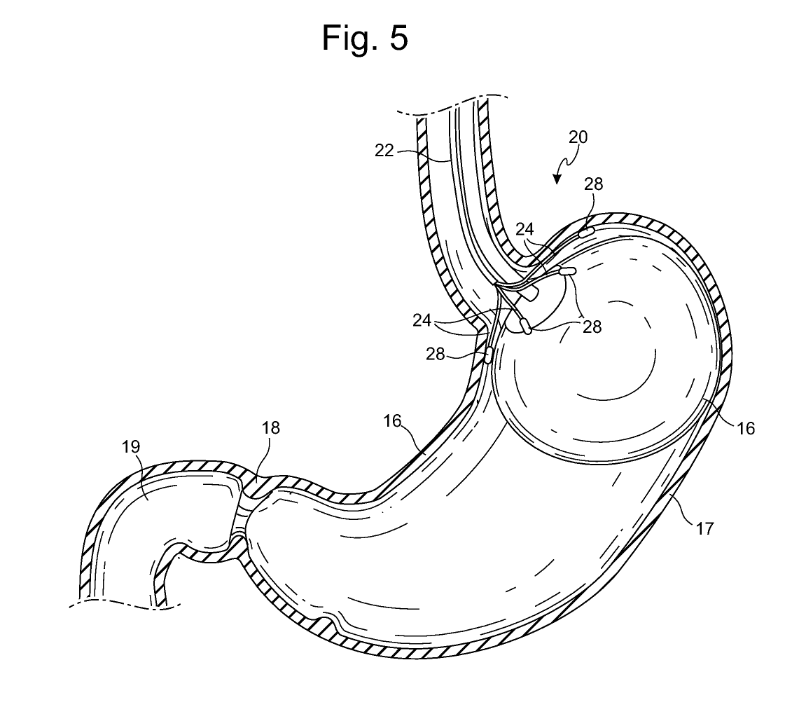

Another use for the embodiment of the instrument depicted in FIG. 3 would be for placing an intragastric balloon 16. See FIG. 5. The balloon would be placed down the esophagus and then filled through a fill tube with saline, air or other fluid to the appropriate volume. Current practice in placing an intragastric balloon 16 is to fill the volume based on the physician's experience or judgment. With this instrument, the sensor 28 could be placed between the balloon 16 and the stomach wall 17 to measure the direct force against the stomach to determine when to stop filling the balloon 16. This would provide a scientific method for adjusting the fill volume of the balloon 16 rather than guessing the appropriate volume. The fill volume would be customized for each patient. The instrument 20 could be used for adjusting the balloon 16 at a later time, by filling or removing fluid in the balloon 16 to customize the fit for each patient over time. In some cases, it may be necessary to increase the balloon fill volume to increase weight loss. It may also be necessary to remove fluid from the balloon 16 to reduce intolerance where a balloon was overfilled at the time of placement. Since the balloon 16 is free to move and rotate within the stomach, it could be monitoring intraluminal and direct patient contact which could be analyzed to detect the difference.

Sensors 28 could be used to gather important patient data to understand performance, positioning, patient status or whether an adjustment needs to be performed for an adjustable bariatric device 10, or whether a bariatric device 10 needs to be replaced or resized. The sensed parameter could detect whether the bariatric device 10 was not in an ideal condition, and display this information to an external controller 86.

Appropriate algorithm(s) may determine and/or control the ideal parameter condition(s), or such condition(s) could be based on a parameter range. For example, the data could be collected from the sensor 28 for a fixed time period. The microprocessor in the external controller 86 could then calculate the average over time, the minimum, the maximum, the standard deviation or the variation in standard deviation over time, or other suitable analysis. Based on the analysis, the microprocessor in the external controller 86 could determine whether the bariatric device 10 was in the ideal position or adjustment state.

Where an accessory 30 with a wireless sensor 28 is used, the sensor monitoring could be performed after placement while the patient is eating or drinking This could also be performed with an instrument 20. The data could be collected, analyzed and used as a guide during the next adjustment. As the patient consumes, the esophageal and stomach peristaltic waves will increase in intensity as they propel the food or drink from the mouth to the stomach. A sensor 28 could detect when these waves increase in amplitude, frequency, and pressure. The parameter detected by the sensor 28 could be read on the external controller by the physician, and then the physician could alter the placement as needed or adjust the bariatric device 10 as needed. The physician could then query the sensor 28 again to determine whether the bariatric device 10 was in the ideal settings.

### Embodiment with Indirect or No Tissue Contact

Another use for this invention would be for placing an intragastric balloon 16. See FIG. 5. As mentioned above, the instrument 20 could be used to monitor the appropriate fill volume of the balloon 16. The instrument 20 could also be used to measure the pressure above in the fundus, around the balloon in the stomach body, or below in the pylorus to monitor intraluminal pressure, instead of direct patient contact pressure or force. Similar to the embodiment depicted in FIG. 4, an accessory 30 could be temporarily attached to the balloon 16 for retrieval later or passing naturally through the GI tract at a later time. The data collected from the sensor 28 would provide a scientific method for adjusting the fill volume of the balloon 16 rather than guessing the appropriate volume. The fill volume would be customized for each patient. The accessory 30 could be used for later filling or removing fluid in the balloon 16 to customize the fit of the balloon over time. In some cases, it may be necessary to increase the balloon fill volume to increase weight loss over time. It may also be necessary to remove fluid from the balloon 16 to reduce intolerance where a balloon was overfilled at the time of placement or over adjusted after placement. Since the balloon 16 is free to move and rotate within the stomach, the sensor 28 could be monitoring intraluminal and direct patient contact. It may be apparent to the user which data is from direct patient contact and indirect patient contact, or alternatively, the data which could be analyzed by the microprocessor to detect the difference.

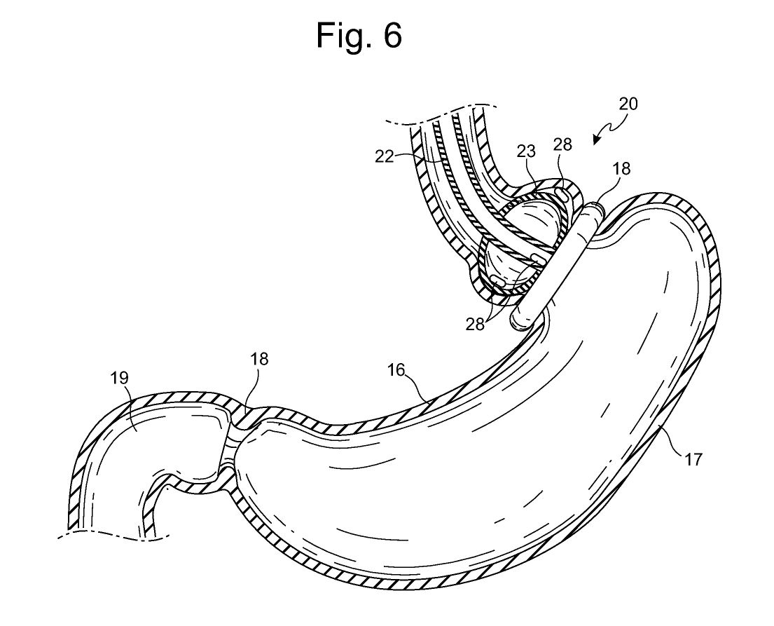

In another embodiment, the instrument 20 could be used when placing a gastric band 18, performing a gastric bypass procedure, or other bariatric surgical procedure. See FIG. 6. The instrument 20 could be introduced down the esophagus and into stomach in the area where the pouch is being created. Although, FIG. 6 shows a gastric band 18, the small pouch could also represent a pouch created during a gastric bypass or other bariatric procedure to create a pouch type structure. The physician could then perform the dissection around the stomach to place the gastric band 18 and lock the band in place. See FIG. 6. The pressure inside the pouch could then be read by the physician prior to suturing the band into place to ensure that an appropriate amount of pressure was being applied. If the pressure were too low or too high, the band 18 could be repositioned or the gastrogastric sutures could be made to reduce or increase the size of the pouch as needed. This instrument 20 could contain a sizing balloon for ease of determining the appropriate pouch size and measuring the intraluminal pressures at the same time. The sensor 28 could be located inside a tube or inside a balloon for indirect measurement, or it could be located on the outside of the balloon or tube for direct patient measurements. There could be one or more sensors 28, or there could be a sensor array 29 to better characterize the pressure profile of the contact surface. This same approach could be taken with a gastric bypass or other bariatric surgical procedure. Prior to making the anastamosis, the pouch area could be clamped with a hemostat or stapler and the pressure measured to ensure the pouch size was appropriate prior to making a permanent cut in the tissue. For the purposes of the claims, “bariatric surgical procedure” includes placement of a gastric band, gastric bypass procedures and variations, sleeve gastrectomy, or other bariatric surgical procedures. The instrument could also be used for adjustments of a gastric band or other adjustable bariatric procedure after the device has been placed and been in situ for awhile. The sensor could detect whether the pressure inside the lumen of the band in the pouch is at an appropriate level, and adjustments could made with the data collected until an desirable level of pressure is achieved.

### Embodiment with Data Recording and Data Transfer

As a variation of the concepts above, the instrument 20 could contain integrated memory, such as a memory module to allow storage of patient and device data. This could include but is not limited to the serial number of the device, the patient's information such as name, patient number, height, weight; the physicians name, the adjustment history including the date and time, the adjustment parameters and the sensed parameters. It could record weight tracking, BMI or other data as needed which could be queried by an external controller. This data could also be transferred into a physician's patient tracking database for ease of patient tracking Similarly, this data could be downloaded and tracked on an internet tracking website, where the patient could log on and see their history and progress. The patient could add information to the website such as weight or an eating log, adverse events or other conditions that the physician or patient would like to track.

### INDUSTRIAL APPLICABILITY

This invention may be industrially applied to the development, manufacture, and use of instruments and accessories for bariatric devices for weight loss purposes.

What is claimed is:

1. A bariatric instrument for sensing one or more parameters in connection with a separate bariatric device, comprising:
a sensor capable of producing signals in response to a parameter within the gastrointestinal tract,
an arm coupled with the sensor,
a central shaft with proximal and distal ends, the distal end coupled with the arm, the central shaft being long enough for the sensor and arm to reach the target area in the approximate location of the separate bariatric device and for the proximal end to be accessible through the mouth of the patient.

2. The bariatric instrument for sensing one or more parameters in connection with a separate bariatric device of claim 1, wherein the sensor does not contact the patient's tissue.

3. The bariatric instrument for sensing one or more parameters in connection with a separate bariatric device of claim 1, wherein the sensor is communicatively coupled with a transmitter capable of transmitting the sensor signals, a receiver capable of receiving signals from an outside source, and a memory module that is capable of storing the signal data from the sensor.

4. The bariatric instrument for sensing one or more parameters in connection with a separate bariatric device of claim 3, further comprising an external controller that communicates with the sensor.

5. The bariatric instrument for sensing one or more parameters in connection with a separate bariatric device of claim 1, further comprising a plurality of sensors, wherein when the sensors are in their operational positions relative to the separate bariatric device, at least one sensor is in contact with the patient's tissues, and at least one sensor is not in contact with the patient's tissues.

6. A sensor accessory for use in connection with an intragastric device, comprising:
a sensor capable of producing signals in response to a parameter within the gastrointestinal tract,
an attachment element coupled with the sensor, said attachment element adapted to couple with an intragastric device.

7. The sensor accessory for use in connection with an intragastric device of claim 6, wherein the sensor does not contact the patient's tissue.

8. The sensor accessory for use in connection with an intragastric device of claim 6, further comprising an arm coupled with at least two sensors.

9. The sensor accessory for use in connection with an intragastric device of claim 6, wherein the sensor is operatively coupled with a transmitter capable of transmitting the sensor signals.

10. The sensor accessory for use in connection with an intragastric device of claim 9, wherein the sensor is operatively coupled with a receiver capable of receiving signals.

11. The sensor accessory for use in connection with an intragastric device of claim 10, wherein the sensor is operatively coupled with a memory module that is capable of storing the signal data from the sensor.

12. The sensor accessory for use in connection with an intragastric device of claim 6, further comprising an external controller, wherein the external controller communicates with the sensor.

13. The sensor accessory for use in connection with an intragastric device of claim 6, further comprising a plurality of sensors, wherein when the sensors are in their operational positions relative to the intragastric device, at least one sensor is in contact with the patient's tissues, and at least one sensor is not in contact with the patient's tissues.

14. An intraluminal instrument for sensing one or more parameters in connection with a bariatric surgical procedure, comprising:
a sensor capable of producing signals in response to a parameter within the gastrointestinal tract, wherein the sensor does not contact the patient's tissue,
an arm coupled with the sensor,
a central shaft with proximal and distal ends, the distal end coupled with the arm, the central shaft being long enough for the sensor and arm to reach the target area in the approximate location of the bariatric surgical procedure and for the proximal end to be accessible through the mouth of the patient.

15. The intraluminal instrument for sensing one or more parameters in connection with a bariatric surgical procedure of claim 14, further comprising a plurality of sensors, wherein when the sensors are in their operational positions relative to the intraluminal device, at least one sensor is in contact with the patient's tissues, and at least one sensor is not in contact with the patient's tissues.
